SEQUENCE LISTING (8 sequences)

SEQ ID NO: 1 - RNA, 20 nt, synthetic construct
  source 1..20: mol_type = other RNA
gtttggtgacttgcgtagag

SEQ ID NO: 2 - RNA, 19 nt, synthetic construct
  source 1..19: mol_type = other RNA
ctcaacagcaacaatggtg

SEQ ID NO: 3 - RNA, 19 nt, synthetic construct
  source 1..19: mol_type = other RNA
ccaattataatgcacaaag

SEQ ID NO: 4 - DNA, 24 bp, Sus scrofa
  source 1..24: mol_type = genomic DNA
gtttggtgacttgcgtagagaggt

SEQ ID NO: 5 - DNA, 28 bp, Sus scrofa
  source 1..28: mol_type = genomic DNA
tctttctcaacagcaacaatggtgtggt

SEQ ID NO: 6 - DNA, 27 bp, Sus scrofa
  source 1..27: mol_type = genomic DNA
aactccaattataatgcacaaagtagg

SEQ ID NO: 7 - RNA, 20 nt, synthetic construct
  source 1..20: mol_type = other RNA
gatgctgcccggtttggcac

SEQ ID NO: 8 - DNA, 98 bp, Sus scrofa
  source 1..98: mol_type = genomic DNA
acgcgaacagcagcgcactcggtgccccgcgcagggtcgcgatgctgcccggtttggcactggtcctgctggccgcctggacggctcgggcgctggag

(19) United States
(12) Patent Application Publication
(10) Pub. No.: US 2024/0172729 A1
(43) Pub. Date: May 30, 2024

(54) GENETICALLY ENGINEERED SWINE FOR PRECLINICAL VALIDATION

(71) Applicants: The Curators of the University of Missouri, Columbia, MO (US); Florida International University, Miami, FL (US); Virginia Tech University, Blacksburg, VA (US)

(72) Inventors: Kiho Lee, Columbia, MO (US); Randall S. Prather, Rocheport, MO (US); Kyungjun Uh, Chungcheongbuk-do (KR); Tim Allen, Miami, FL (US); Tim Jarome, Blacksburg, VA (US)

(21) Appl. No.: 18/482,521

(22) Filed: Oct. 6, 2023

Related U.S. Application Data
(60) Provisional application No. 63/378,711, filed on Oct. 7, 2022.

(51) Int. Cl. A01K 67/0276; C12N 9/22; C12N 15/113

(52) U.S. Cl. CPC A01K 67/0276; C12N 9/22; C12N 15/113; A01K 2217/075; A01K 2217/15; A01K 2227/108; A01K 2267/0312; C12N 2310/20

(57) ABSTRACT

Disclosed are genetically modified swine having a genetic modification in at least one of a Presenilin 1 (PSEN1) gene and an amyloid beta precursor protein (APP) gene and method for creating the genetically modified swine.

### CROSS REFERENCE TO RELATED APPLICATIONS

This application claims priority under 35 U.S.C. § 119(e) to U.S. Provisional Patent Application Ser. No. 63/378,711, filed on Oct. 7, 2022, the disclosure of which is hereby incorporated by reference in its entirety.

### STATEMENT OF GOVERNMENT SUPPORT

This invention was made with government support under OD011140, and AG079292 awarded by the National Institutes of Health. The government has certain rights in the invention.

### INCORPORATION OF A SEQUENCE LISTING

A computer readable form of a Sequence Listing containing the file named “23UMC031USb.xml”, created on Feb. 15, 2024, which is 8,210 bytes in size as measured in MICROSOFT WINDOWS® EXPLORER), is provided herein and is herein incorporated by reference. This Sequence Listing consists of SEQ ID NOs:1-8.

### BACKGROUND

The present disclosure relates generally to medicine. More particularly, the compositions and methods are particularly suitable for investigating human diseases.

Alzheimer's Disease (AD) is the leading cause of dementia in the United States. AD patients with an inherited form of the disease carry mutations in the presenilin proteins (PSEN1; PSEN2) or in the amyloid precursor protein (APP). The disease-linked mutations result in increased production of the longer form of amyloid-beta (main component of amyloid deposits found in AD brains). Presenilins are postulated to regulate APP processing through their effects on gamma-secretase, which cleaves APP.

PSEN1 encodes the catalytic subunit of the gamma-secretase enzyme, which breaks down amyloid beta precursor protein (APP) into smaller subunits, including beta-amyloid (Aβ) peptides. Mutations of PSEN1 increase the production of Aβ2, the more toxic form that is prone to aggregation and formation of the dense, insoluble oligomers known as “plaques” that are a hallmark pathology of AD.

The APP gene encodes a cell surface receptor and transmembrane precursor protein that is cleaved by secretases to form a number of peptides. Some of these peptides are secreted and can bind to the acetyltransferase complex APBB1/TIP60 to promote transcriptional activation, while others form the protein basis of the amyloid plaques found in the brains of patients with Alzheimer disease. In addition, two of the peptides are antimicrobial peptides, having been shown to have bacteriocidal and antifungal activities. Mutations in this gene have been implicated in autosomal dominant Alzheimer disease and cerebroarterial amyloidosis (cerebral amyloid angiopathy). Multiple transcript variants encoding several different isoforms have been found for this gene.

While progress has been made using rodent AD models, there is a need to validate large animal models of AD to better understand the disease process in animals that are more like humans. Pigs have long been regarded as the ideal preclinical model for drug development in biomedical research due to their substantial similarities to humans across major organ systems, and are increasingly favored in translational neuroscience (e.g., traumatic brain injury, neurotoxicity). However, pig AD models have yet to be generated that reflect human behavioral symptoms.

Accordingly, there exists a need for large animal models of AD. The present disclosure addresses this need by providing pig models of AD by manipulating the Presenilin-1 gene (PSEN1) and/or amyloid precursor protein (APP) because human mutations of these genes are the most common cause of early-onset familial AD (FAD).

### BRIEF DESCRIPTION

In one aspect, the present disclosure is directed to a genetically modified swine comprising a genetic modification in a Presenilin 1 (PSEN1) gene and a genetic modification in an amyloid beta precursor protein (APP) gene.

In one aspect, the present disclosure is directed to a genetically modified swine comprising a genetic modification in a PSEN1 gene.

In one aspect, the present disclosure is directed to a genetically modified swine comprising a genetic modification in APP.

In one aspect, the present disclosure is directed to a method of creating a genetically modified swine comprising a genetic modification in a Presenilin 1 (PSEN1) gene and a genetic modification in an amyloid beta precursor protein (APP) gene.

In one aspect, the present disclosure is directed to a method of creating a genetically modified swine comprising a genetic modification in a Presenilin 1 (PSEN1) gene.

In one aspect, the present disclosure is directed to a method of creating a genetically modified swine comprising a genetic modification in an amyloid beta precursor protein (APP) gene.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a graph depicting memory testing in wild type and PSEN1 genetically-modified pigs in a custom-built automated T-maze task run. Dashed lines represent +/−1 SEM.

FIGS. 2A and 2B depict experimental designs to disrupt PSEN1 (FIG. 2A) and APP (FIG. 2B) using CRISPR.

### DETAILED DESCRIPTION

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are described below.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

Unless defined otherwise, all technical and scientific terms used herein have the same meaning as commonly understood by one of skill in the art to which the invention pertains. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.” The terms “comprising”, “including” and “having” are intended to be inclusive and mean that there may be additional elements other than the listed elements. The term “about” means up to ±10%.

The present disclosure is directed to animals and methods for producing gene edited animals having modifications of a PSEN1 gene and an APP gene. The present disclosure is also directed to animals and methods for producing gene edited animals having modifications of a PSEN1 gene. The present disclosure is directed to animals and methods for producing gene edited animals having modifications of an APP gene. As used herein, “gene editing,” “gene edited” “genetically edited” and “gene editing effectors” refer to the use of homing technology with naturally occurring or artificially engineered nucleases, also referred to as “molecular scissors, “homing endonucleases,” or “targeting endonucleases.” The nucleases create specific double-stranded chromosomal breaks (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganucleases (e.g., meganucleases re-engineered as homing endonucleases). The terms also include the use of transgenic procedures and techniques, including, for example, where the change is a deletion or relatively small insertion (typically less than 20 nt) and/or does not introduce DNA from a foreign species. The term also encompasses progeny animals such as well as those created by sexual crosses or asexual propagation from the initial gene edited animal. These animals can be created using any of a number of protocols which make use of gene editing technologies such as the Clustered Regularly Interspaced Short Palindromic Repeats (CRISPR)/Cas system, Transcription Activator-Like Effector Nucleases (TALENs), Zinc Finger Nucleases (ZFN), PRIME editing, recombinase fusion proteins, leucine zippers, and meganucleases. The term “CRISPR” stands for “clustered regularly interspaced short palindromic repeats.” The term “Cas9” refers to “CRISPR associated protein 9.” The terms “CRISPR/Cas9” or “CRISPR/Cas9 system” refer to a programmable nuclease system for genetic engineering that includes a Cas9 protein, or derivative thereof, and one or more non-coding RNAs that can provide the function of a CRISPR RNA (crRNA) and trans-activating RNA (tracrRNA) for the Cas9. The crRNA and tracrRNA can be used individually or can be combined to produce a “guide RNA” (gRNA). The crRNA or gRNA provide sequence that is complementary to the genomic target. CRISPR/Cas9 systems are described further herein below.

As used herein, “knock-out” means disruption of the structure or regulatory mechanism of a gene. Knock-outs may be generated through gene editing technologies, homologous recombination of targeting vectors, replacement vectors or hit-and-run vectors or random insertion of a gene trap vector resulting into complete, partial or conditional loss of gene function. The term “knock-in” means replacement of an endogenous gene with a transgene or with same endogenous gene with some structural modification(s), but retaining the transcriptional control of the endogenous gene.

The term “animal” includes any non-human animal, for example a domestic animal (e.g. a livestock animal). The term “livestock animal” includes any animals traditionally raised in livestock farming, for example a porcine animal, a bovine animal (e.g., beef of dairy cattle), an ovine animal, a caprine animal, an equine animal (e.g., horses or donkeys), buffalo, camels, or an avian animal (e.g., chickens, turkeys, ducks, geese, guinea fowl, or squabs). This term “livestock animal” does not include rats, mice, or other rodents.

As used herein “operably linked” includes reference to a functional linkage between two nucleic acid sequences, e.g., a promoter sequence and a second sequence, wherein the promoter sequence initiates and mediates transcription of the DNA sequence corresponding to the second sequence. Generally, operably linked means that the nucleic acid sequences being linked are contiguous and, where necessary join two protein coding regions, contiguously and in the same reading frame.

“Wild type” means those animals and blastocysts, embryos or cells derived therefrom, that have not been genetically edited or otherwise genetically modified and are usually inbred and outbred strains developed from naturally occurring strains.

As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

Methods of alignment of sequences for comparison are well-known in the art. Optimal alignment of sequences for comparison may be conducted by the local homology algorithm of Smith and Waterman, Adv. Appl. Math. 2: 482(1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); and by computerized implementations of these algorithms, including, but not limited to: CLUSTAL in the PC/Gene program by Intelligenetics, Mountain View, California; GAP, BESTFIT, BLAST, FASTA, and TFASTA, and related programs in the GCG Wisconsin Genetics Software Package, Version 10 (available from Accelrys Inc., 9685 Scranton Road, San Diego, California, USA). The CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al, Computer Applications in the Biosciences 8: 155-65 (1992), and Pearson, et al, Methods in Molecular Biology 24: 307-331 (1994).

The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol, 215: 403-410 (1990); and, Altschul et al., Nucleic Acids Res. 25: 3389-3402 (1997). Software for performing BLAST analyses is publicly available, for example through the National Center for Biotechnology Information (ncbi.nlm.nih.gov/). This algorithm has been thoroughly described in a number of publications. See, e.g., Altschul S F et al, Gapped BLAST and PSI-BLAST: a new generation of protein database search programs, 25 NUCLEIC ACIDS RES. 3389 (1997); National Center for Biotechnology Information, THE NCBI HANDBOOK [INTERNET], Chapter 16: The BLAST Sequence Analysis Tool (McEntyre J, Ostell J, eds., 2002), available at http://www.ncbi.nlm.nih.gov/books/NBK21097/pdf/ch16.pdf. The BLASTP program for amino acid sequences has also been thoroughly described (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Nat'l. Acad. Sci. USA 90: 5873-5877 (1993)). A number of low-complexity filter programs can be employed to reduce such low-complexity alignments. For example, the SEG (Wooten and Federhen, Comput. Chem., 17: 149-163 (1993)) and XNU (Claverie and States, Comput. Chem., 17: 191-201 (1993)) low-complexity filters can be employed alone or in combination.

As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions may be calculated according to the algorithm of Meyers and Miller, Computer Applic. Biol. Sci., 4: 11-17 (1988), for example as implemented in the program PC/GENE (Intelligenetics, Mountain View, California, USA). As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The present disclosure is directed to animals and methods for producing gene edited animals having modifications of a PSEN1 gene and an APP gene. The present disclosure is also directed to animals and methods for producing gene edited animals having modifications of a PSEN1 gene. The present disclosure is directed to animals and methods for producing gene edited animals having modifications of an APP gene. The edited chromosomal sequence (a PSEN1 gene and/or an APP gene) may be (1) inactivated, (2) modified, or (3) comprise an integrated sequence resulting in a null mutation. Thus, a genetically edited animal comprising an inactivated chromosomal sequence may be termed a “knock out” or a “conditional knock out.” Similarly, a genetically edited animal comprising an integrated sequence may be termed a “knock in” or a “conditional knock in.” Furthermore, a genetically edited animal comprising a modified chromosomal sequence may comprise a targeted point mutation(s) or other modification such that an altered protein product is produced. Briefly, the process can comprise introducing into an embryo or cell at least one RNA molecule encoding a targeted zinc finger nuclease and, optionally, at least one accessory polynucleotide. The method further comprises incubating the embryo or cell to allow expression of the zinc finger nuclease, wherein a double-stranded break introduced into the targeted chromosomal sequence by the zinc finger nuclease is repaired by an error-prone non-homologous end-joining DNA repair process or a homology-directed DNA repair process. The method of editing chromosomal sequences encoding a protein associated with germline development using targeted zinc finger nuclease technology is rapid, precise, and highly efficient. Alternatively, the process can comprise using a CRISPR/Cas9 system to modify the genomic sequence. To use Cas9 to modified genomic sequences, the protein can be delivered directly to a cell, an mRNA that encodes Cas9 can be delivered to a cell, or a gene that provide for expression of an mRNA that encodes Cas9 can be delivered to a cell. In addition, either target specific crRNA and a tracrRNA can be delivered directly to a cell or target specific gRNA(s) can be to a cell (these RNAs can alternatively be produced by a gene constructed to express these RNAs). Selection of target sites and designed of crRNA/gRNA are well known in the art.

Provided herein are non-human animals, offspring of said animals, and animal cells comprising at least one modified chromosomal sequence in a gene encoding PSEN1 protein and/or at least one modified chromosomal sequence in a gene encoding APP protein. In any of the animals, offspring or cells, the modified chromosomal sequence can comprise an insertion in the gene encoding the PSEN1 protein, a deletion in the gene encoding the PSEN1 protein, an insertion in the gene encoding the APP protein, a deletion in the gene encoding the APP protein, and combinations thereof. The insertion or deletion can cause PSEN1 protein and/or APP protein production or activity to be reduced, as compared to PSEN1 protein and/or APP protein production or activity in an animal, offspring, or cell that lacks the insertion or deletion.

Where the animal, offspring, or cell comprises a porcine animal, offspring, or cell, the modified chromosomal sequence can comprise a modification in exon 9 of the gene encoding the PSEN1 protein, an intron that is contiguous with exon 9 of the gene encoding the PSEN1 protein, or a combination thereof. The modified chromosomal sequence suitably comprises a modification in the intron upstream of exon 9 in combination with a modification in exon 9 of the gene encoding the PSEN1 protein. Where the animal, offspring, or cell comprises a porcine animal, offspring, or cell, the modified chromosomal sequence can comprise a modification in exon 1 of the gene encoding the APP protein. A particularly suitable exemplary embodiment includes animals and cells having a modified chromosomal sequence with a modification in the intron upstream of exon 9 in combination with a modification in exon 9 of the gene encoding the PSEN1 protein and a modification in exon 1 of the gene encoding the APP protein. A particularly suitable exemplary embodiment includes animals and cells having a modified chromosomal sequence with a modification in the intron upstream of exon 9 in combination with a modification in exon 9 of the gene encoding the PSEN1 protein. A particularly suitable exemplary embodiment includes animals and cells having a modified chromosomal sequence with a modification in exon 1 of the gene encoding the APP protein.

Various other techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. Cloning, for instance, may be used to make a mosaic animal if its cells are modified at the blastocyst state, or genomic modification can take place when a single-cell is modified. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

For embryo/zygote microinjection, a nucleic acid construct or mRNA is introduced into a fertilized egg; 1 or 2 cell fertilized eggs are used as the nuclear structure containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18 gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO2. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 400,000 sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO2 atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs or mRNA can be injected into one of the pronuclei or into the cytoplasm. Then the injected eggs can be transferred to a recipient female (e.g., into the oviducts of a recipient female) and allowed to develop in the recipient female to produce the transgenic or gene edited animals. The embryos can be injected with using an Eppendorf FEMTO JET injector and can be cultured until blastocyst formation. Rates of embryo cleavage and blastocyst formation and quality can be recorded.

Embryos can be surgically transferred into uteri of asynchronous recipients. Typically, 40-60 embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic or gene edited cell such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp bevelled tip is used to inject the transgenic or gene edited cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al. (1998) Science 280, 1256-1258 and U.S. Pat. Nos. 6,548,741, 7,547,816, 7,989,657, or 6,211,429. For pigs, recipient females can be checked for pregnancy approximately 20-21 days after transfer of the embryos.

Standard breeding techniques can be used to create animals that are homozygous for the inactivated gene from the initial heterozygous founder animals. Homozygosity may not be required, however. Gene edited pigs described herein can be bred with other pigs of interest.

Once gene edited animals have been generated, inactivation of an endogenous nucleic acid can be assessed using standard techniques. Initial screening can be accomplished by PCR-Sanger sequencing using genomic DNA and also with cDNA. Polymerase chain reaction (PCR) techniques also can be used in the initial screening PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News 12,1; Guatelli et al. (1990) Proc. Natl. Acad. Sci. USA 87:1874; and Weiss (1991) Science 254:1292. At the blastocyst stage, embryos can be individually processed for analysis by PCR, Southern hybridization and splinkerette PCR (see, e.g., Dupuy et al. Proc Natl Acad Sci USA (2002) 99:4495).

A method of inactivating a gene in a genetically edited animal comprises inducing RNA interference against a target gene and/or nucleic acid such that expression of the target gene and/or nucleic acid is reduced. In vitro cells, in vivo cells, or a genetically edited animal such as a livestock animal that express an RNAi directed against a gene encoding CD163 can be used. The RNAi may be, for instance, selected from the group consisting of siRNA, shRNA, dsRNA, RISC and miRNA.

An inducible system may be used to inactivate a PSEN1 gene and/or an APP gene. Various inducible systems are known that allow spatial and temporal control of inactivation of a gene. Several have been proven to be functional in vivo in porcine animals. An example of an inducible system is the tetracycline (tet)-on promoter system, which can be used to regulate transcription of the nucleic acid. The Cre/lox system uses the Cre recombinase, which catalyzes site-specific recombination by crossover between two distant Cre recognition sequences, i.e., loxP sites. A DNA sequence introduced between the two loxP sequences (termed floxed DNA) is excised by Cre-mediated recombination.

A variety of nucleic acids may be introduced into cells for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7(3): 187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

In one aspect, the present disclosure is directed to a genetically modified swine comprising a deletion in a Presenilin 1 (PSEN1) gene and a deletion in an amyloid beta precursor protein (APP) gene. Suitable embodiments of deletions in the PSEN1 gene to combine with the deletion of the APP gene include a genetic modification resulting in a deletion of exon 9 of the PSEN1 gene, a genetic modification to an intron 5′ upstream of exon 9 of the PSEN1 gene resulting in a deletion of exon 9 of the PSEN1 gene, a genetic modification in a region of the PSEN1 gene that includes an intron 5′ upstream of exon 9 and exon 9 of the PSEN1 gene resulting in a deletion of exon 9 of the PSEN1 gene. Suitable embodiments of deletions in the APP gene to combine with the deletion of the PSEN1 gene include a genetic modification in exon 1 of the APP gene resulting in a deletion of the APP gene.

Suitably, the genetically modified swine comprising the deletion in a Presenilin 1 (PSEN1) gene and the deletion in an amyloid beta precursor protein (APP) gene is a PSEN1 null and an APP null swine. Suitably, the genetically modified swine is a PSEN1^{+/-} and an APP^{+/-} swine. Suitably, the genetically modified swine is a PSEN1^{-/-} and an APP^{-/-} swine. Suitably, the genetically modified swine is a PSEN1^{-/-} and an APP^{-/-} swine. Suitably, the genetically modified swine is a PSEN1^{+/-} and an APP^{-/-} swine. The genetic modification to the PSEN1 gene and the APP gene results in a complete lack of production of the PSEN1 protein and/or APP protein or an PSEN1 protein and/or APP protein that does not function properly.

In one aspect, the present disclosure is directed to a genetically modified swine comprising a deletion in a Presenilin 1 (PSEN1) gene. Suitable embodiments of deletions in the PSEN1 gene include a genetic modification resulting in a deletion of exon 9 of the PSEN1 gene, a genetic modification to an intron 5′ upstream of exon 9 of the PSEN1 gene resulting in a deletion of exon 9 of the PSEN1 gene, a genetic modification in a region of the PSEN1 gene that includes an intron 5′ upstream of exon 9 and exon 9 of the PSEN1 gene resulting in a deletion of exon 9 of the PSEN1 gene. Suitable embodiments of deletions in the APP gene to combine with the deletion of the PSEN1 gene include a genetic modification in exon 1 of the APP gene resulting in a deletion of the APP gene.

Suitably, the genetically modified swine comprising the deletion in a Presenilin 1 (PSEN1) gene is a PSEN1 null swine. Suitably, the genetically modified swine is a PSEN1^{+/-} (heterozygous) swine. Suitably, the genetically modified swine is a PSEN1^{-/-} (homozygous) swine. The genetic modification to the PSEN1 gene results in a complete lack of production of the PSEN1 protein or an PSEN1 protein that does not function properly.

In one aspect, the present disclosure is directed to a genetically modified swine comprising a deletion in an amyloid beta precursor protein (APP) gene. Suitable embodiments of deletions in the APP gene include a genetic modification in exon 1 of the APP gene resulting in a deletion of the APP gene.

Suitably, the genetically modified swine comprising the deletion in an amyloid beta precursor protein (APP) gene is an APP null swine. Suitably, the genetically modified swine is an APP^{+/-} (heterozygous) swine. Suitably, the genetically modified swine is an APP^{-/-} (homozygous) swine. The genetic modification to the APP gene results in a complete lack of production of the APP protein or an APP protein that does not function properly.

In another aspect, the present disclosure is directed to creating a genetically modified swine comprising a deletion in a Presenilin 1 (PSEN1) gene and a deletion in an amyloid beta precursor protein (APP) gene. The method includes introducing a guide RNA targeting a PSEN1 gene into a zygote to introduce double strand breaks in the PSEN1 gene, introducing a guide RNA targeting an APP gene into the zygote to introduce double strand breaks in the APP gene, culturing the zygote, transferring the cultured zygote to a recipient female; and allowed to develop in the recipient female to produce the genetically modified swine.

In another aspect, the present disclosure is directed to creating a genetically modified swine comprising a deletion in a Presenilin 1 (PSEN1) gene. The method includes introducing a guide RNA targeting a PSEN1 gene into a zygote to introduce double strand breaks in the PSEN1 gene, culturing the zygote, transferring the cultured zygote to a recipient female; and allowed to develop in the recipient female to produce the genetically modified swine.

In another aspect, the present disclosure is directed to creating a genetically modified swine comprising a deletion in an amyloid beta precursor protein (APP) gene. The method includes introducing a guide RNA targeting an APP gene into the zygote to introduce double strand breaks in the APP gene, culturing the zygote, transferring the cultured zygote to a recipient female; and allowed to develop in the recipient female to produce the genetically modified swine.

A method of breeding to create animals or lineages that have a deletion in a Presenilin 1 (PSEN1) gene and a deletion in an amyloid beta precursor protein (APP) gene is also provided. The method comprises genetically modifying an oocyte or a sperm cell to introduce a modified chromosomal sequence in a gene encoding PSEN1 protein and genetically modifying the oocyte or the sperm cell to introduce a modified chromosomal sequence in a gene encoding an APP protein into at least one of the oocyte and the sperm cell, and fertilizing the oocyte with the sperm cell to create a fertilized egg containing the modified chromosomal sequence in the gene encoding a PSEN1 protein and the gene encoding an APP protein. Alternatively, the method comprises genetically modifying a fertilized egg to introduce a modified chromosomal sequence in a gene encoding a PSEN1 protein and to introduce a modified chromosomal sequence in a gene encoding an APP protein into the fertilized egg. The method further comprises transferring the fertilized egg into a surrogate female animal, wherein gestation and term delivery produces a progeny animal, screening the progeny animal reduced expression of PSEN1 and reduced expression of APP, and selecting progeny animals that have reduced expression of PSEN1 and reduced expression of APP as compared to animals that do not comprise a modified chromosomal sequence in a gene encoding a PSEN1 protein and do not comprise a modified chromosomal sequence in a gene encoding an APP protein.

A method of breeding to create animals or lineages that have a deletion in a Presenilin 1 (PSEN1) gene is also provided. The method comprises genetically modifying an oocyte or a sperm cell to introduce a modified chromosomal sequence in a gene encoding PSEN1 protein into at least one of the oocyte and the sperm cell, and fertilizing the oocyte with the sperm cell to create a fertilized egg containing the modified chromosomal sequence in the gene encoding a PSEN1 protein. Alternatively, the method comprises genetically modifying a fertilized egg to introduce a modified chromosomal sequence in a gene encoding a PSEN1 protein into the fertilized egg. The method further comprises transferring the fertilized egg into a surrogate female animal, wherein gestation and term delivery produces a progeny animal, screening the progeny animal for PSEN1 expression, and selecting progeny animals having reduced PSEN1 expression as compared to animals that do not comprise a modified chromosomal sequence in a gene encoding a PSEN1 protein.

A method of breeding to create animals or lineages that have a deletion in an amyloid beta precursor protein (APP) gene is also provided. The method comprises genetically modifying an oocyte or a sperm cell to introduce a modified chromosomal sequence in a gene encoding an APP protein into at least one of the oocyte and the sperm cell, and fertilizing the oocyte with the sperm cell to create a fertilized egg containing the modified chromosomal sequence in the gene encoding an APP protein. Alternatively, the method comprises genetically modifying a fertilized egg to introduce a modified chromosomal sequence in a gene encoding an APP protein into the fertilized egg. The method further comprises transferring the fertilized egg into a surrogate female animal, wherein gestation and term delivery produces a progeny animal, screening the progeny animal for APP expression, and selecting progeny animals having reduced APP expression as compared to animals that do not comprise a modified chromosomal sequence in a gene encoding a APP protein.

### EXAMPLES

### Behavior Assessment

Spatial working memory was tested in wild type (WT) and PSEN1 genetically-modified pigs in a custom-built automated T-maze task run using overhead tracking cameras, custom code, and automated guillotine doors. Spatial working memory is considered a gold-standard test and sensitive measure of dementia-related cognitive decline for preclinical Alzheimer's disease models.

A pig model lacking functional PSEN1 was created by using a CRISPR/Cas9 system and were validated for cognitive and molecular characteristics of AD. Animals were tested in a fully automated T-maze apparatus (17′×13′) designed to tax spatial working memory in which pigs were rewarded for correctly alternating choices at the T-junction. The evaluations focused on the delayed spatial alternation task, which forced pigs to wait in the starting area during randomized delays (from 5 seconds to 240 seconds) and burdened spatial working memory. The T-maze task took advantage of pigs' natural tendency to alternate choices in a food rewarded paradigm (sometimes called a win-shift strategy) and required animals to turn the opposite way from their last choice at the T-junction. Each correct alternation was rewarded with a food pellet automatically dispensed into food bowels (right and left side). Memory was tested by imposing delays randomly from 5 to 240 seconds between choices. During delays pigs were restricted to the enclosed start area. As delays increased memory burden increased. Analyses were restricted to sessions in which delays were randomly presented at 5, 60, 120 and 240 seconds and included multiple sessions for each pig and reflected steady performance levels. Delays could not be anticipated and were equally likely to occur on any given trial (sampling with replacement).

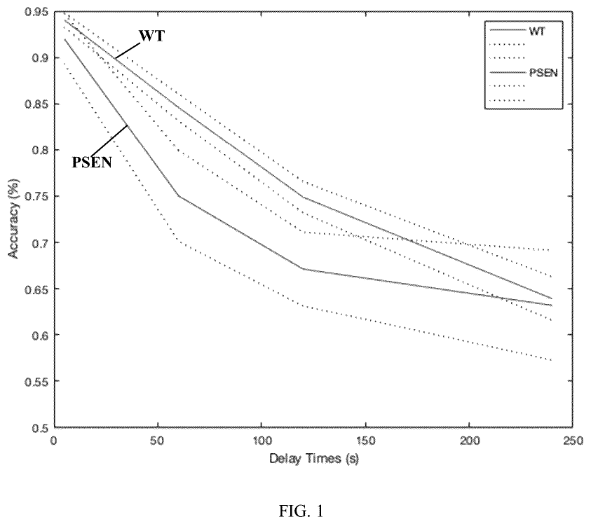

WT (blue line) and PSEN1 (red line) pigs showed a high level of accuracy (>90%) after short 5 second delays (FIG. 1). There were no differences at this short delay comparing WT to PSEN1 pigs (p>0.05) suggesting perceptual, motivational, and motor functions were all intact. No differences observed in PSEN1 and control pigs at the shortest delays suggested that the animals were similarly capable and motivated to alternate for the food reward. However, PSEN7 pigs were significantly impaired at longer delays (60 and 120 second conditions (WT versus PSEN1, p's<0.05)), demonstrating a spatial working memory deficit. These data showed that PSEN1 pigs had dementia-like memory deficits. Performance in both pig floored at 240 seconds. Dashed lines represent +/−1 SEM. This pattern of results is reminiscent of memory deficits in rodent AD models and in mild-to-moderate stages of AD human patients.

### CRISPR Design to Disrupt PSEN1 and APP

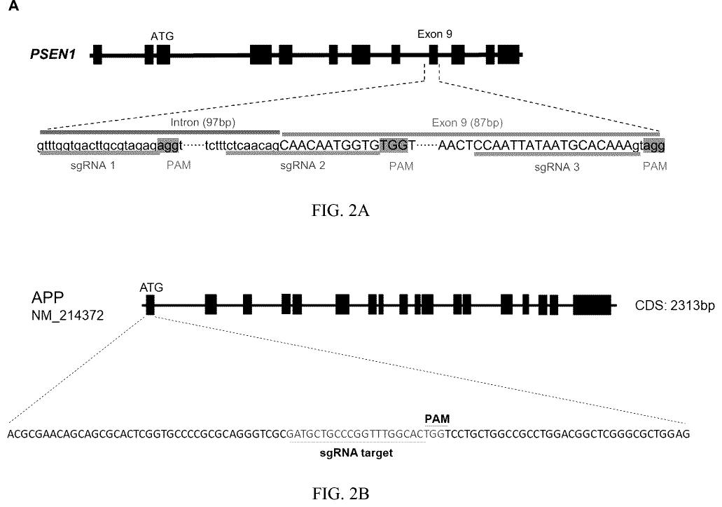

To disrupt PSEN1, three sgRNAs targeting different regions of PSEN1 gene (SEQ ID NO:4, SEQ ID NO:5, SEQ ID NO:6) were designed using the CRISPR design tool, CRISPOR. To induce a targeted deletion of exon 9 in PSEN1, two sgRNA combinations were used—sgRNA 1 (SEQ ID NO:1) and sgRNA 3 (SEQ ID NO:3) and sgRNA 2 (SEQ ID NO:2) and sgRNA 3 (SEQ ID NO:3) (Table 1; FIG. 2A). The combinations were designed to introduce double strand breaks flanking exon 9 of PSEN1. Each sgRNA combinations (10 ng/μl each) were mixed with in vitro-synthesized Cas9 mRNA (20 ng/μl), were injected into the cytoplasm of presumable zygotes after in vitro fertilization (IVF) using the FemtoJet microinjector (Eppendorf). Embryos were microinjected in manipulation medium on a heated stage of a Nikon inverted microscope. After the microinjection, the zygotes were washed and then cultured in PZM3 media for 5-6 days.

TABLE 1
PSEN1 Guide RNA and PSEN1 Target Sequence.

| Guide RNA | SEQ ID NO | Target Sequence | SEQ ID NO |
|---|---|---|---|
| sgRNA1 | SEQ ID NO: 1 | gtttggtgacttgcgtagagaggt | SEQ ID NO: 4 |
| gtttggtgacttgcgtagag | | | |
| sgRNA2 | SEQ ID NO: 2 | tctttctcaacagcaacaatggtgtggt | SEQ ID NO: 5 |
| ctcaacagcaacaatggtg | | | |
| sgRNA3 | SEQ ID NO: 3 | aactccaattataatgcacaaagtagg | SEQ ID NO: 6 |
| ccaattataatgcacaaag | | | |

An identical approach as described for PSEN1 was used to design APP CRISPR sgRNA (SEQ ID NO:4) and produce the pigs (Table 2; FIG. 2B).

TABLE 2
APP Guide and APP Target Sequence.

| | Sequence | SEQ ID NO |
|---|---|---|
| APP sgRNA | GATGCTGCCCGGTTTGGCAC | SEQ ID NO: 7 |
| APP gene | ACGCGAACAGCAGCGCACTCGGTGCCCCGCGCAG | SEQ ID NO: 8 |
| target | GGTCGCGATGCTGCCCGGTTTGGCACTGGTCCTGC | |
| sequence | TGGCCGCCTGGACGGCTCGGGCGCTGGAG | |

These examples establish pigs as a model species for behavioral neuroscience and specifically towards translational goals to better understand AD progression, pathology, and therapeutic interventions.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

What is claimed is:

1. A genetically modified swine comprising a genetic modification in a Presenilin 1 (PSEN1) gene and a genetic modification in an amyloid beta precursor protein (APP) gene.

2. The genetically modified swine of claim 1, wherein the genetic modification in the PSEN1 gene is a deletion in the PSEN1 gene selected from the group consisting of a genetic modification resulting in a deletion of exon 9 of the PSEN1 gene, a genetic modification to an intron 5′ upstream of exon 9 of the PSEN1 gene resulting in a deletion of exon 9 of the PSEN1 gene, a genetic modification in a region of the PSEN1 gene that includes an intron 5′ upstream of exon 9 and exon 9 of the PSEN1 gene resulting in a deletion of exon 9 of the PSEN1 gene, and combinations thereof.

3. The genetically modified swine of claim 1, wherein the genetic modification in the APP gene is a deletion in exon 1 of the APP gene.

4. The genetically modified swine of claim 1, wherein the deletion in the PSEN1 gene results in a complete lack of production of PSEN1 protein or an PSEN1 protein that does not function.

5. The genetically modified swine of claim 1, wherein the deletion in the APP gene results in a complete lack of production of APP protein or an APP protein that does not function.

6. A genetically modified swine comprising a genetic modification in a PSEN1 gene.

7. The genetically modified swine of claim 6, wherein the genetic modification in the PSEN1 gene is selected from the group consisting of a deletion of exon 9 of the PSEN1 gene, a genetic modification to an intron 5′ upstream of exon 9 of the PSEN1 gene resulting in a deletion of exon 9 of the PSEN1 gene, a genetic modification in a region of the PSEN1 gene that includes an intron 5′ upstream of exon 9 and exon 9 of the PSEN1 gene resulting in a deletion of exon 9 of the PSEN1 gene, and combinations thereof.

8. The genetically modified swine of claim 6, wherein the deletion in the PSEN1 gene results in a complete lack of production of PSEN1 protein or an PSEN1 protein that does not function properly.

9. A genetically modified swine comprising a genetic modification in an APP gene.

10. The genetically modified swine of claim 9, wherein the genetic modification is a deletion in exon 1 of the APP gene.

11. The genetically modified swine of claim 9, wherein the deletion in the APP gene results in a complete lack of production of APP protein or an APP protein that does not function properly.

12. A method of creating a genetically modified swine comprising a genetic modification in a Presenilin 1 (PSEN1) gene and a genetic modification in an amyloid beta precursor protein (APP) gene, the method comprising: introducing a guide RNA targeting a PSEN1 gene into a zygote to introduce double strand breaks in the PSEN1 gene to result in a deletion in the PSEN1 gene; introducing a guide RNA targeting an APP gene into the zygote to introduce double strand breaks in the APP gene to result in a deletion in the APP gene; culturing the zygote; transferring the cultured zygote to a recipient female; and allowing the zygote to develop in the recipient female to produce the genetically modified swine.

13. The method of claim 12, wherein the genetic modification in the PSEN1 gene is selected from the group consisting of a genetic modification resulting in a deletion of exon 9 of the PSEN1 gene, a genetic modification to an intron 5′ upstream of exon 9 of the PSEN1 gene resulting in a deletion of exon 9 of the PSEN1 gene, a genetic modification in a region of the PSEN1 gene that includes an intron 5′ upstream of exon 9 and exon 9 of the PSEN1 gene resulting in a deletion of exon 9 of the PSEN1 gene, and combinations thereof.

14. The method of claim 12, wherein the deletion in the PSEN1 gene results in a complete lack of production of PSEN1 protein or an PSEN1 protein that does not function properly and wherein the deletion in the APP gene results in a complete lack of production of APP protein or an APP protein that does not function properly.

15. A method of creating a genetically modified swine comprising a deletion in a Presenilin 1 (PSEN1) gene, the method comprising: introducing a guide RNA targeting a PSEN1 gene into a zygote to introduce double strand breaks in the PSEN1 gene to result in a deletion in the PSEN1 gene; culturing the zygote; transferring the cultured zygote to a recipient female; and allowing the zygote to develop in the recipient female to produce the genetically modified swine.

16. The method of claim 15, wherein the genetic modification in the PSEN1 gene is selected from the group consisting of a genetic modification resulting in a deletion of exon 9 of the PSEN1 gene, a genetic modification to an intron 5′ upstream of exon 9 of the PSEN1 gene resulting in a deletion of exon 9 of the PSEN1 gene, a genetic modification in a region of the PSEN1 gene that includes an intron 5′ upstream of exon 9 and exon 9 of the PSEN1 gene resulting in a deletion of exon 9 of the PSEN1 gene, and combinations thereof.

17. The method of claim 15, wherein the deletion in the PSEN1 gene results in a complete lack of production of PSEN1 protein or an PSEN1 protein that does not function properly.

18. A method of creating a genetically modified swine comprising a genetically modified swine comprising a deletion in an amyloid beta precursor protein (APP) gene, the method comprising: introducing a guide RNA targeting an APP gene into the zygote to introduce double strand breaks in the APP gene to result in a deletion in the APP gene; culturing the zygote; transferring the cultured zygote to a recipient female; and allowing the zygote to develop in the recipient female to produce the genetically modified swine.

19. The method of claim 18, wherein the deletion in the APP gene is a genetic modification in exon 1 of the APP gene.

20. The method of claim 18, wherein the deletion in the APP gene results in a complete lack of production of APP protein or an APP protein that does not function properly.
